(19) United States
(12) Patent Application Publication
(10) Pub. No.: US 2014/0349980 A1
(43) Pub. Date: Nov. 27, 2014

(54) DELAYED-RELEASE GLUCOCORTICOID TREATMENT OF RHEUMATOID ARTHRITIS BY IMPROVING SIGNS AND SYMPTOMS, SHOWING MAJOR OR COMPLETE CLINICAL RESPONSE AND BY PREVENTING FROM JOINT DAMAGE

(71) Applicant: Achim Schäffler, Beerfelden (DE)

(72) Inventor: Achim Schäffler, Beerfelden (DE)

(21) Appl. No.: 14/305,165

(22) Filed: Jun. 16, 2014

Related U.S. Application Data
(63) Continuation of application No. 13/791,816, filed on Mar. 8, 2013, now abandoned.
Application No. 13/791,816 is a continuation of application No. 12/697,384, filed on Feb. 1, 2010, now abandoned.
(60) Provisional application No. 61/148,435, filed on Jan. 30, 2009.

(51) Int. Cl. A61K 31/573

(52) U.S. Cl. CPC A61K 31/573
USPC 514/171; 514/179

(57) ABSTRACT

The present invention refers to the treatment of a patient suffering from rheumatoid arthritis by showing a reduction in signs and symptoms, a major or complete clinical response (remission) or even prevention of structural damages to the joints by administering a delayed-release dosage form of a glucocorticoid to a subject in need thereof.

The present invention refers to the treatment of a patient suffering from rheumatoid arthritis by showing a reduction in signs and symptoms, a major or complete clinical response (i.e. by showing remission) or even prevention from structural damages to the joints by administering a delayed-release dosage form of a glucocorticoid to a subject in need thereof.

### BACKGROUND OF THE INVENTION

### Role of Low-Dose Corticoid Therapy in Clinical Practice

Diseases of rheumatoid nature like rheumatoid arthritis (RA) are chronic, autoimmune disorders in which inflammation of the synovial joint lining is accompanied by joint pain and stiffness and usually leads to bone and joint destruction, deformity, disability, and even death. RA affects about 1% of the population and is 2 to 3 times more common in women than in men (CPMP/EWP/556/95). Early diagnosis, suppression of inflammation, and aggressive treatment strategies are regarded as important requisites for a favorable outcome (Pincus 2005). Glucocorticoids are widely used to treat the disease and are often administered in combination with other drugs, especially disease-modifying antirheumatic drugs (DMARDs) and non-steroidal anti-inflammatory drugs (NSAIDs) (Bijisma 2003). Prednisone, prednisolone and methylprednisolone are among the most common glucocorticoids for the treatment of RA.

Use and types of oral corticoid RA therapy differ according to region and published estimates vary. According to one source, in 2002 about 40 to 50% of patients in France, Germany, Italy and Spain received such therapy compared to about 20% in the United Kingdom (UK). Prednisone was the most common corticoid in France, Italy and Spain (94%, 59% and 43% of treated patients, respectively) whereas prednisolone was the most common in Germany and the UK (50 and 100%, respectively). A study in 150 patients who attended a US clinic during the period 1999 to 2001 showed that 144 (96%) patients took prednisone in combination with DMARDs (86%) or alone (10%) (Pincus 2005).

Glucocorticoids have a broad spectrum of anti-inflammatory and immunosuppressive effects. They act by inhibiting leukocyte traffic; interfering with functions of leukocytes, fibroblasts, and endothelial cells; and suppressing the synthesis and actions of inflammatory cytokines including interleukin-6 (IL-6) (Buttgereit 2005). When they were first introduced, glucocorticoids were administered to RA patients for long periods at high doses exceeding 10 mg/day prednisone or equivalent. These high-dose, long-term regimens were highly effective but were associated with pleiotropic effects and unacceptable adverse reactions. This led to the development of low-dose regimens in order to reduce the incidence of side effects and optimized the benefit:risk ratio (Buttgereit 2005). High corticoid doses are now only considered suitable for short-term therapy in special cases (e.g. for treatment of a severe flare of RA). Decreases in prescribed corticoid dose are illustrated by an evaluation of patients who attended a US clinic between 1984 and 1986 (1985 cohort) or between 1999 and 2001 (2000 cohort) (Pincus 2005). The mean prednisone dose was 7.8 mg/day in 1985 compared to 4 mg/day in 2000, with median doses of 5 and 4 mg/day, respectively.

Long-term, low-dose, corticoid therapy (defined as daily doses of ≦10 mg prednisone or equivalent) is currently recognized as an important part of standard treatment for RA (ACR guideline, Conn 2001). Below 10 mg the daily dose should be decreased stepwise until the lowest, still effective dose for disease control is reached. In addition to providing immediate relief of symptoms such as morning stiffness and pain, the low-dose corticoid regimen also prevents progression of disease. Several randomized studies performed since the mid-1990s have shown that low-dose prednis(ol)one slows the rate of joint damage (as measured by radiographic images) in patients with early, active RA. In a double-blind, placebo-controlled study, 7.5 mg/day prednisolone reduced joint destruction when given for 2 years in combination with other standard RA treatments (Kirwan 1995). When prednisolone was stopped, joint destruction returned to the same level as in the control group (Hickling 1998). In a more recent double-blind, placebo-controlled study, prednisone (10 mg/day) slowed progression of joint damage over periods of 2 and 5 years in patients who had not been pretreated with DMARDs (van Everdingen 2002, Jacobs 2005). In an open-label, DMARD-controlled study (Svensson 2005), prednisolone at doses of 7.5 mg/day, decreased radiographic progression when given in combination with DMARDs for 2 years. However, Capell et al could not confirm these positive findings of immediate release prednisone on the prevention of structural damages (Capell 2004).

Thus, the effects of glucocorticoids on signs and symptoms and major or complete clinical response (remission) or to prevent from structural damages to the joints in rheumatoid arthritis patients remain unclear. Signs and symptoms, remission and prevention from structural damages in rheumatoid arthritis patients are defined in the Guidance for Industry; Clinical Development Programs for Drugs, Devices, and Biological Products for the Treatment of Rheumatoid Arthritis (RA), February 1999, the content of which is herein incorporated by reference.

Safety of Low-Dose Long-Term Corticoid Therapy

Soon after glucocorticoids were introduced for the treatment of RA in the 1950s it became apparent that long-term use of high doses was associated with clinically significant side effects that included osteoporosis, glucose intolerance, infections, peptic ulcers and gastrointestinal bleeding, cataracts and glaucoma, as well as atherosclerotic disease. Several clinical studies and literature reviews have been performed to assess the safety profile of low-dose, long-term corticoid therapy. It is generally agreed that side effects can be reduced by using as low a dose as possible for each individual patient. One study that compared RA patients with and without prednisone treatment concluded that long-term prednisone use at doses ≧5 mg/day was associated with the dose-dependent development of specific AEs (Saag 1994). However, this study was retrospective with historical case controls and included prednisone doses up to 15 mg/day. A working group of rheumatologists and experts from other therapeutic areas has recently conducted a comprehensive literature review of the adverse effects of low-dose (≦10 mg/day prednisolone equivalent), long-term glucocorticoid therapy by a primary search of textbooks and review papers (da Silva 2006). Their review also included analysis of data from 4 prospective, randomized, controlled studies in which prednisolone (5 to 10 mg/day) was given to RA patients for 2 years (Capel/2004, Kirwan 1995, van Everdingen 2002, Wassenberg 2005). Common side effects seen at high doses were not observed at low doses or were less frequent. The experts concluded that “the overall fear of glucocorticoid toxicity in RA, as quoted in textbooks and review articles, is probably overestimated based on observations with higher dose therapy. The balance of risks and benefits of low-dose therapy dearly differs from that of medium- and high-dose therapy . . . ” Osteoporosis, obesity, hypertension, family history of diabetes or glaucoma were listed as risk factors requiring more careful observation. In addition to osteoporosis, adverse effects that may need regular checks were defined as Cushingoid syndrome, adrenal crisis of corticoid withdrawal, new onset of diabetes mellitus, worsening of glycemia control in patients with diabetes mellitus, cataracts, glaucoma, peptic ulcer (in combination with NSAIDs), and hypertension.

Delayed-Release Prednisone Tablets

Patients with active RA suffer from clinical signs and symptoms that include joint stiffness, pain, and swelling. Patients have assessed these symptoms (and related factors such as disability and mobility) as being important outcomes of RA treatment (Ahlmen et al. 2005, Carr et al. 2003, Hewlett et al. 2005). Clinical symptoms vary during the day and are more severe early in the morning after awakening than in the afternoon or evening (Cutolo et al. 2003, Cutolo and Masi 2005). Indeed, morning stiffness is such a typical symptom of RA that it has become a standard diagnostic criterion for the disease (Arnett et al. 1988, ACR Guideline 2002).

The mechanisms responsible for the circadian variation of RA symptoms are complex and involve the HPA axis and endogenous inflammatory mediators. Inflammation causes increased production of inflammatory cytokines. In comparison with healthy subjects, RA patients therefore have higher serum concentrations of interleukins (IL), especially IL-6, and tumor necrosis factor-alpha (TNF-α) and levels display a pronounced circadian rhythm, with higher night-time concentrations that peak at 02:00 to 06:00 (Arvidson et al. 1994; Crofford 1997; Cutolo 2003, 2005).

Increased levels of IL-6 are produced in response to inflammation but IL-6 is a potent activator of the HPA axis and stimulates the release of cortisol from the adrenal cortex to counteract the inflammation (Cutolo 2005, Mastorakos 2000). In RA patients, it seems that the response of the permanently stimulated HPA axis is inadequate and levels of endogenous cortisol are insufficient to combat the inflammation (Gudbjörnsson 1996). Administration of exogenous glucocorticoids acts—among other therapeutic effects—as a replacement therapy and supplements the inadequate levels of endogenous cortisol (Cutolo 2005).

Endogenous cortisol and exogenous therapeutic glucocorticoids inhibit the synthesis of IL-6 and other pro-inflammatory cytokines. In this context, prednis(ol)one and methylprednisolone are ideally suited exogenous corticoid due to its comparatively short half-life of 3-4 h. Low-dose oral prednis(ol)one or methylprednisolone are usually given for symptomatic relief as a single morning dose to minimize potential interference with the HPA axis. However, in order to provide optimal relief of morning stiffness and joint pain it has been proposed that the drug should be given shortly before the expected nocturnal increase of IL-6. A randomized study has investigated the efficacy of standard IR (Immediate Release) low-dose prednisolone (5 or 7.5 mg/day) given at 02:00 or at 07:30 for 4 days in 26 patients with active RA who were being treated with standard anti-rheumatic drugs (predominantly NSAIDs) but who had not received glucocorticoids in the 3 months before the study (Arvidson et al. 1997). Night-time administration of prednisolone at 02:00 resulted in highly statistically significant improvements in morning stiffness, joint pain, as well as suppression of serum concentrations of IL-6 (p<0.01). Much smaller effects (p<0.05) were only observed for morning stiffness and IL-6 concentrations after conventional morning dosing at 07:30. The authors concluded that low doses of glucocorticoids improved acute RA symptoms if they were administered before the circadian flare of increased IL-6 synthesis and inflammatory activity. However, it remained unclear what would happen to the patients if they would be treated for a longer period of time.

Karatay et al investigated in 2002 the administration of an IR low-dose prednisone tablet over a period of 6 months at 02:00 vs 07:30. The results were disappointing because a difference in morning stiffness could not be observed. One explanation of this could be that the short term effects observed by Arvidson disappear after several days or weeks of therapy. Thus, the effects on long term night time administration of glucocorticoids remained unclear.

Furthermore, all patients in both study (Arvidson 1997; Karatay 2002) were corticoid naïve. Thus, the question has arisen, how low-dose night-time prednisone would work in patients already pre-treated with low-dose corticoids and what would happen if they would get the night-time dose over a longer time with a higher compliance rate.

Although administration of glucocorticoids at 02:00 resulted in improved efficacy in one of two studies, in practice this would be highly inconvenient for the patient and likely to result in poor quality of sleep and/or compliance.

Buttgereit et al. (2008) and U.S. Ser. No. 11/833,322 disclose in a Phase 3 study on the effects of Delayed-Release prednisone on morning stiffness. After 3 months of treatment with Prednisone Delayed-Release no improvement over immediate release prednisone was shown for the American College of Rheumatology (ACR) 20/50/70 responder rate.

U.S. Pat. No. 5,792,476 describes a pharmaceutical composition for peroraladministration for rheumatoid arthritis, which comprises a glucocorticoid as active ingredient and which leads to release in the small intestine. The composition is a granulate which is laminated with an inner layer which is resistant to a pH of 6.8, and with an outer layer which is resistant to a pH of 1.0.

U.S. Pat. No. 6,488,960 describes a pharmaceutical dosage form for controlled release of corticoids, reference being made to the formulations described in U.S. Pat. No. 5,792,476.

WO 01/08421 describes a tablet having a core which is coated by at least two layers, one of which completely encloses the other. The coating layers can be produced by spray coating and/or pressing.

WO 01/68056 discloses a pharmaceutical preparation having a release profile with a time delay, comprising a core and at least one hydrophilic or lipophilic coating surrounding the core, where the coating is slowly swollen, dissolved, eroded or changed in its structure in another way through the water present in the release medium, so that the core or parts of the core become accessible to the release medium. The coating may be formed for example as pressed coating.

WO 02/072034 discloses a pharmaceutical dosage form for delayed release, having a core which comprises as active ingredient a glucocorticoid and a material which brings about delayed release and includes at least one natural or synthetic gum.

WO 2004/093843 discloses a tablet with a specific core geometry to release the active ingredient in a specific delayed release manner.

WO 2006/027266 discloses a pharmaceutical dosage form with site and time controlled gastrointestinal release of an active agent, particularly a corticosteroid. The pharmaceutical dosage form is preferably a coated tablet having a core comprising the corticosteroid and a swellable/disintegration adjuvant, and an inert outer coating. The coating is compressed at a pressure chosen to result in the release of the corticosteroid at a predetermined position in the gastrointestinal tract.

U.S. Ser. No. 11/833,322 discloses the treatment of a rheumatic disease and/or osteoarthritis by administering a delayed-release dosage form of a glucocorticoid to a subject in need thereof.

### SUMMARY OF THE INVENTION

The present inventors have carried out a clinical study in order to further test the efficacy of a delayed-release prednisone tablet. It was surprisingly found that the administration, particularly the long-term administration of the delayed-release prednisone tablet shows a significant response to ACR20/50/70 (FDA guidance for industry, 1999).

ACR 20/50/70 Responders were defined as patients suffering from rheumatoid arthritis, who after administration of a delayed-release glucocorticoid dosage form show an improvement from baseline to endpoint (12 weeks) which fulfils all 3 of the following criteria:

(i) ≧20%, 50% or 70% reduction in the tender joint count (0-28).

(ii) ≧20%, 50% or 70% reduction in the swollen joint count (0-28).

(iii) ≧20%, 50% or 70% reduction in at least 3, e.g. 3, 4 or 5 of 5 of the following additional measures:
Patient assessment of pain (100 mm visual analogue scale [VAS]).
Patient's global assessment of disease activity (VAS).
Physician's global assessment of disease activity (VAS).
Functional Disability Index of the Health Assessment Questionnaire (HAQ-DI).
C-reactive protein (CRP) or erythrocyte sedimentation rate (ESR) as acute-phase reactant. CRP was used. If the CRP result was not available then the ESR result was used to calculate the ACR20 responder status

Besides the significant ACR20/50/70 response, delayed-release prednisone showed also a positive effect on bone markers osteocalcin and urine CTX I, which was completely unexpected.

Thus, the invention refers to a method for the treatment of a patient suffering from rheumatoid arthritis showing an reduction in signs and symptoms, major or complete clinical response (remission) and even prevent from structural damages to the joints, which comprises administering to said patient an effective amount of a glucocorticoid contained in a delayed-release dosage form, wherein said treatment is administered once daily for at least about two weeks.

The invention further refers to a method for the treatment of a patient for the prevention from structural damages to the joints in rheumatoid arthritis, which comprises administering to said patient an effective amount of glucocorticoid contained in a delayed-release dosage form, wherein said treatment is administered once daily for at least about two weeks.

The invention further refers to a method for the treatment of a patient suffering from rheumatoid arthritis with minor or incomplete clinical response after prior treatment with another medicament, which comprises administering to said patient an effective amount of a glucocorticoid contained in a delayed-release dosage form, wherein said treatment is administered once daily for at least about two weeks.

The invention further refers to a method for the treatment of a patient suffering from signs and symptoms of rheumatoid arthritis, which comprises administering to said patient an effective amount of a glucocorticoid contained in a delayed-release dosage form, wherein said treatment is administered once daily for at least about two weeks.

The invention further refers to a method for the treatment of a patient suffering from rheumatoid arthritis which comprises administering to said patient an effective amount of a glucocorticoid contained in a delayed-release dosage form, wherein the pharmacokinetics after administering of said dosage form are equivalent to the pharmacokinetics after administering an immediate release dosage form formulation, wherein the pharmacokinetics include an equivalent Cmax, an equivalent AUC and/or an equivalent tmax-tlag.

The invention further refers to a method for the treatment of a patient suffering from joint destruction accompanied by, caused by or associated with rheumatoid arthritis, which comprises administering to said patient an effective amount of a glucocorticoid contained in a delayed-release dosage form, wherein said treatment is administered once daily for at least about 6 months.

According to an aspect of the invention the delayed-release dosage form may be administered to

(i) patients with severe diseases,

(ii) patients with moderate diseases,

(iii) patients with mild diseases,

(iv) patients with short disease duration (<2 years),

(v) patients with mid-term disease duration (2-5 years) or

(vi) patients with long-lasting disease duration (>5 years).

According to a further aspect of the present invention the delayed-release dosage form may be administered to

i patients with severe, long lasting morning stiffness;

ii patients with moderate morning stiffness;

iii patients with mild morning stiffness;

iv patients with severe, long lasting pain;

v patients with moderate pain; or

vi patients with mild pain.

According to still a further aspect of the present invention the delayed-release dosage form may be administered to

i patients with high Interleukin 6 levels;

ii patients with medium Interleukin 6 levels or

iii patients with low Interleukin 6 levels.

According to still a further aspect of the present invention the delayed-release dosage form may be administered to

i patients with minor clinical response to another medicament or

ii patients with incomplete clinical response to another medicament in order to achieve remission.

According to still a further aspect of the present invention the delayed-release dosage form may be administered for prevention of structural damage to the joints in

iii patients with diagnosed disease and no measurable joint destruction

iv patients at an early stage of joint destruction or

v patients at an advanced stage of joint destruction.

According to still a further aspect of the present invention the delayed-release dosage form may be administered to the
patients who have been pre-treated with an immediate release dosage form of a glucocorticoid,
ii patients who are refractory to treatment with an immediate release dosage form of a glucocorticoid, or
iii glucocorticoid naïve patients.

According to still a further aspect of the present invention the delayed-release dosage form may be administered to
i patients who have been pre-treated with other medicaments like a NSAID, a DMARD, a TNFα inhibitor, an IL-6 inhibitor and/or an analgetic agent, or
ii patients who have not been pre-treated with any other meddicaments like a NSAID, a DMARD, a TNFα inhibitor, an IL-6 inhibitor and/or an analgetic agent.

According to still a further aspect of the present invention the delayed-release dosage form may be administered in combination with at least one further medicament which is a NSAID, a DMARD, a TNFα inhibitor, an IL-6 inhibitor and/or an analgetic agent.

According to still a further aspect of the present invention, the pharmacokinetic behaviour of the glucocorticoid contained in the delayed-release dosage form is equivalent to the pharmacokinetics after administering an immediate release dosage form, i.e. an equivalent Cmax, an equivalent AUC and/or an equivalent tmax-tlag value.

Still a further aspect of the present invention is a method for the treatment of a patient suffering from incomplete clinical response after prior treatment and/or from accelerated joint destruction of underlying rheumatoid arthritis, which comprises administering to said patient an effective amount of a glucocorticoid contained in a delayed-release dosage form, wherein said treatment is administered once daily for at least about 3 months (12 weeks).

### DETAILED DESCRIPTION OF THE INVENTION

The present invention refers to the use of a delayed-release dosage form of a glucocorticoid. The release of the active ingredient is preferably delayed for a time period of 2-10 hours after intake, preferably 2-6, more preferably 3-5 hours after intake the active ingredient may be released in the upper sections of the intestine and/or in the lower sections of the intestine. More preferably, the active ingredient is released in the upper sections of the intestine within a period of 2-6 hours. The delayed-release dosage form is preferably administered to the patient at or before bedtime, more preferably in the evening, e.g. from about 9:00 pm to about 11:00 pm. Because inflammation is accompanied with circadian fluctuations in the concentration of pro-inflammatory cytokines (such as Interleukin-6) which peaks during sleeping hours, bedtime administration allows an efficacious concentration of the active ingredient to be present when such concentration peaks.

The delayed-release dosage form can be any kind of dosage form, like a capsule or a tablet. It is preferably a tablet, e.g. as described in WO 2006/027266, which is herein incorporated by reference. The dosage form preferably comprises
a) a core having at least one glucocorticoid-active ingredient and having at least one swellable adjuvant and/or a disintegrant such that the active Ingredient is rapidly released from the dosage form when the core is contacted with gastrointestinal fluids, and
b) an inert, e.g. a non-soluble and non-swellable coating pressed onto the core, said coating being capable of preventing substantial release of the active ingredient for a defined time period following ingestion of the dosage form.

The inert coating initially prevents release of the active ingredient or the active ingredient combination over an exactly defined period, so that no absorption can occur. The water present in the gastrointestinal tract penetrates slowly in through the coating and, after a time which is previously fixed by the pressure for compression, reaches the core. The coating ingredients show neither swelling nor diluting of parts of the coating. When the core is reached, the water penetrating in is very rapidly absorbed by the hydrophilic ingredients of the core, so that the volume of the core increases greatly and, as a consequence thereof, the coating completely bursts open, and the active ingredient and the active ingredient combination respectively is released very rapidly.

A particularly advantageous embodiment of this press-coated delayed-release tablet is achieved when a previously compressed core tablet is subsequently compressed with a multilayer tablet press to a press-coated tablet.

The tablet coating typically consists of the following materials in order to achieve a delayed release profile:
polymer or copolymer of acrylic acid, methacrylic acid etc. (e.g. Eudragits or Carbopol),
cellulose derivatives such as hydroxypropylmethylcellulose, hydroxypropylcellulose, carboxymethylcellulose, ethylcellulose, cellulose acetate,
polyvinyl alcohol,
polyethylene glycol,
salts of higher fatty acids, esters of monohydric or polyhydric alcohols with short-, medium- or long-chain, saturated or unsaturated fatty acids. Specifically, stearic acid triglycerides (e.g. Dynersan) or glycerol behenate (e.g. Compritol) are used.

In addition, further adjuvants should also be added to these materials so that the tablet coating can be compressed. Typically used here are fillers such as lactose, various starches, celluloses and calcium hydrogen phosphate or dibasic calcium phosphate. The glidant used is normally magnesium stearate, and in exceptional cases also talc and glycerol behenate. A plasticizer is often also added to the coating material, preferably from the group of polyethylene glycol, dibutyl phthalate, diethyl citrate or triacetin.

In order to achieve an optimal release profile, the tablet core must also fulfil certain tasks and exhibit certain properties. Thus, after the lag phase has elapsed, a rapid release profile is achieved if typical disintegrants are added to the inner core; which are derived for example from the group of the following substances: cellulose derivatives, starch derivatives, crosslinked polyvinylpyrrolidone. The use of a blowing agent, for example resulting from a combination of a weak acid and a carbonate or bicarbonate, may also promote rapid release. The tablet core typically consists additionally of matrix or filling ingredients (e.g. lactose, cellulose derivatives, calcium hydrogen phosphate or other substances known from the literature) and lubricant or glidant (usually magnesium stearate, in exceptional cases also talc and glycerol behenate).

The size of the core tablet preferably should not exceed 6 mm (preferably 5 mm) in diameter, because otherwise the press-coated tablet becomes too large for convenient ingestion. As a result thereof, the dosages of the active ingredients are in the range from 0.1 to 50 mg, very particularly between 1 and 20 mg.

The in vitro release profile of the dosage form according to the invention is preferably such that less than 5% of the active ingredient is released during the lag phase. After the release phase has started, preferably ≧80%, particularly preferably ≧90%, of the active ingredient is released within one hour. More preferably, the delayed-release dosage form has a dissolution time of equal to or less than about 2 hours after the lag time has been reached). The in vitro release is preferably determined using the USP paddle dissolution model in water.

The employed active ingredients are derived from the group of glucocorticoids and all show comparable physicochemical properties. Such include cortisone, hydrocortisone, prednisone, prednisolone, methylprednisolone, budesonide, dexamethasone, fludrocortisone, fluocortolone, cloprednole, deflazacort, triamcinolone, or the corresponding pharmaceutically acceptable salts and/or esters thereof. This applies in particular to prednisone, prednisolone, methylprednisolone, budesonide, dexamethasone, fluocortolone, cloprednole, and deflazacort or the corresponding pharmaceutically acceptable salts and/or esters thereof.

In the present case of the delayed-release tablet, the following combination of core materials and coating materials has proved to be particularly suitable for achieving a time- and site-controlled release with exclusion of pH and food influences:

The coating preferably comprises:
hydrophobic, waxy substances with an HLB value of less than about 5, preferably around 2. Carnauba wax, paraffins, cetyl ester waxes are preferably employed therefor. Glycerol behenate has proved to be particularly suitable. The use of about 20-60%, in particular about 30-50%, in the coating has proved to be very advantageous;
non-fatty, hydrophobic filling materials such as calcium phosphate salts, e.g. dibasic calcium phosphate. The use of about 25-75% of these filling materials, in particular of about 40-60%, in the coating has proved to be very advantageous here;
in addition, the tablet coating preferably also consists of binders, e.g. polyvinylpyrrolidone (PVP), typically in concentrations of about 4-12%, specifically about 7-10%, and glidants such as magnesium stearate, in concentrations of about 0.1-2%, in the specific case of about 0.5-1.5%. Colloidal silicon dioxide can for example be used as flow regulator, normally in concentrations of about 025-1%. In addition, to distinguish different dosages, a colorant can be added to the tablet coating, preferably an iron oxide pigment in concentrations of about 0.001-1%.

The core tablet preferably comprises:
an active ingredient or an active ingredient combination from the group of glucocorticoids, preferably prednisone, prednisolone, methylprednisolone, budesonide, dexamethasone, fludrocortisone, fluocortolone, cloprednole, deflazacort, and triamcinolone, and the corresponding salts and esters thereof. The dosages of the active ingredients are in the region of about 0.1-50 mg, very especially between about 1 and 20 mg;
in addition, the core tablet preferably comprises a filler such as, for example, lactose, starch derivatives or cellulose derivatives. Lactose is preferably employed. The filler is typically present in concentrations of about 50-90%, specifically of about 60-80%. A disintegrant is additionally present and is typically crosslinked PVP or sodium carboxymethylcellulose, typically in concentrations of about 10-20%. It is additionally possible for a binder, e.g. PVP, to be present, typically in concentrations of about 2-10%, specifically of about 5.5-9%, and a lubricant such as magnesium stearate, in concentrations of about 0.1-2%, in the specific case of about 0.5-1.5%. Colloidal silicon dioxide is normally used as flow regulator, normally in concentrations of about 0.25-1%. It is additionally possible, for visually distinguishing the core from the coating, to add a colorant, preferably an iron oxide pigment in concentrations of about 0.01-1%.

Preferably, the delayed-release dosage form is administered as a long-term treatment to a subject in need thereof for a time sufficient to reduce and/or abolish the disease and/or disease symptoms. The long term treatment usually comprises daily administration of the medicament for an extended period of time, e.g. for at least two weeks, preferably for at least 4 weeks, more preferably for at least 8 weeks, even more preferably for at least 12 weeks, and most preferably for at least 6 months or at least 12 months.

According to the present invention refers to the novel treatment of groups of patients suffering from rheumatic arthritis. These patient groups are selected from:
i patients with severe diseases characterized by a Disease Activity Score (DAS) of >5.1 (Le Loet 2006) and/or a Physicians Assessment;
ii patients with moderate diseases characterized by a Disease Activity Score (DAS) of >3.2 but <5.1 and/or a Physicians Assessment;
iii patients with mild diseases characterized by a Disease Activity Score (DAS) of <3.2 and/or a Physicians Assessment;
iv patients with short disease duration of less than 2 years,
v patients with mid-term disease duration of 2-5 years, and
vi patients with long-lasting disease duration of more than 5 years.

Further patient groups may be selected from:
i patients with severe, long lasting morning stiffness characterized by a duration of morning stiffness >180 min,
ii patients with moderate morning stiffness between 100 and 180 min,
iii patients with mild morning stiffness of less than 100 min,
iv patients with severe, long lasting pain characterized by a VAS scale with >70 mm,
v patients with moderate pain characterized by a VAS scale with >50-70 mm,
vi patients with mild pain characterized by a VAS scale with <50 mm.

Further patient groups may be selected from:
i patients with high Interleukin 6 levels, e.g. more than 3000 IU/l;
ii patients with medium Interleukin 6 levels, e.g. between 3000 and 1000 IU/l;
iii patients with low Interleukin 6 levels, e.g. less than 1000 IU/l.

Further patient groups may be selected from:
i patients who have been pre-treated with an immediate release dosage form of a glucocorticoid;
ii patients who are refractory to treatment with an immediate-release dosage form of a glucocorticoid, and
iii glucocorticoid naive patients.

Further patient groups may be selected from:
i patients with minor clinical response to another medicament, or
ii patients with incomplete clinical response to another medicament, wherein the other medicament may be an immediate-release form of a glucocorticoid or a medicament, e.g. selected from an NSAID, a DMARD, a TNFα inhibitor, an analgetic agent and/or an Interleukin 6 inhibitor.

Further patient groups may be selected from:
iii patients with diagnosed disease and no measurable joint destruction;
iv patients who are at the early stage of joint destruction; and
v patients who are at an advanced stage of joint destruction.

Further patient groups may be selected from:
i patients who have been pre-treated with other medicaments like an NSAID, a DMARD, a TNFα inhibitor, an Interleukin 6 inhibitor and/or an analgetic agent, and
ii patients who have not been pre-treated with any other medicaments like a NSAID, a DMARD, a TNFα inhibitor, an Interleukin 6 inhibitor and/or an analgetic agent.

By means of administering a delayed-release tablet, the daily dose of the glucocorticoid may be substantially reduced compared to an immediate-release tablet of the glucocorticoid. Thus, the disease-inhibiting effect may be obtained by a significantly lower dose of the active ingredient, whereby the occurrence and/or intensity of site effect is diminished. For example, the daily dose of the glucocorticoid can be reduced by at least 10%, more preferably by at least 20%, e.g. by 10-50% compared to an immediate-release tablet. Thus, the reduced daily dose of prednis(ol)one in Prednisone delayed-release is preferably in the range of 1 to 5 mg/day compared to 6-10 mg/day for a standard IR tablet.

The treatment according to the present invention may comprise the treatment of a rheumatoid arthritis without any further medicament. On the other hand, the invention may comprise the treatment of a rheumatic arthritis in combination with at least one further medicament which is preferably selected from the groups of NSAIDs, DMARDs, TNF α inhibitors, IL-6 inhibitors, analgetic agents or combinations thereof. Especially preferred is a combination with Tarenflurbil.

NSAIDs are preferably selected from arylalkanoic acids (Diclofenac, Indometacin, Sulindac) from 2-arylpropionic acids (Flurbiprofen, Carprofen, Fenoprofen, Flurbiprofen, Ibuprofen, Ketoprofen, Ketorolac, Laxoprofen, Naproxen, Tiaprofenic acid), from N-arylanthranilic acids (Mefenamic acid, Meclofenamic acid), from Oxicams (Piroxicam, Meloxicam) or from Coxibs (Celecoxib, Parecoxib, Etoricoxib) or from combinations thereof. Especially preferred is a combination with Tarenflurbil.

DMARDs are preferably selected from gold preparations, chloroquine, azathioprine, sulfasalazine, cyclophosphamide, penicillamine, hydroxychloroquine, methotrexate, thorium dioxide suspension, levamisole, cyclosporin, interferone, leflunomide, TNFα inhibitor, an Interleukin-1, an Interleukin-6 inhibitor or from combinations thereof.

TNF α inhibitors and IL-1 inhibitors are preferably selected from antibodies or soluble receptors such as etanercept, inflixima, anakinra, adalimumab and from cominations thereof.

IL-6 inhibitors are preferably selected from antibodies or soluble receptors such as tocilizumab.

Analgetic agents are preferably selected from salicylates (Aspirin, Methyl salicylate, Diflunisal, Benorylate, Faislamine, Amoxiprin), from pyrazolidine derivatives (Phenylbutazone, Oxyphenylbutazone) or paracetamol or from combinations thereof.

The dose of the at least one further medicament may be substantially reduced e.g. by at least 10%, preferably by at least 20%, e.g. by 10-50%. Alternatively, the first usage of TNF α inhibitors or IL-6 inhibitors can be postponed to a later point in time.

The present invention particularly refers to the treatment of rheumatoid arthritis. Based on the results of the clinical trials described in the present application, it is evident that the delayed-release dosage form of a glucocorticoid, particularly a long-term treatment, is of therapeutic benefit. The administration of the delayed-release dosage form is effective without having undesired side effects.

The dose of the glucocorticoid may vary during the course of treatment. For example, the patient may be administered a relatively high dose during the initiation of therapy (e.g., about 10-40 mg/day or higher of prednisone, or an equivalent amount of another glucocorticoid), which may be reduced downward over a period of time (e.g., over 3-4 weeks) according to the patient's response, to a maintenance therapy dose of about 10 mg/day or less of prednisone, or an equivalent amount of another glucocorticoid. Alternatively, the patient may be started on a relatively low dose, which may be adjusted upward over a period of time (e.g., over 3-4 weeks) to a maintenance therapy dose of about 7.5 to 10 mg/day of prednisone, or an equivalent amount of another glucocorticoid.

By using the Delayed-Release Prednisone formulation therapy can be initiated with 5 mg/day or below.

In a particular embodiment of the present invention the pharmacokinetic behaviour after administering the delayed-release dosage form is equivalent to the pharmacokinetcis after administering an immediate-release glucocorticoid dosage form, particularly a formulation of the same dosage of the same glucocorticoid. An equivalent pharmacokinetic behaviour may include an equivalent maximum plasma concentration (Cmax), i.e. a Cmax which is about 80 to about 125%, more particularly about 90 to about 110% of the Cmax of the corresponding immediate-release formulation. An equivalent pharmacokinetic behaviour may also include an equivalent AUC, which may be about 80 to about 125%, particularly about 90 to about 110% of the AUC of the corresponding immediate-release formulation. Further, the equivalent pharmacokinetics may include an equivalent tmax-tlag value for the delayed release and the immediate release formulation, particularly about 1 to 4 hours, more particularly about 2 to 3 hours, wherein tmax is the time after administration when Cmax is reached. Tlag corresponds to the in vivo lag-time for the release of the delayed-release dosage form. For an immediate-release dosage form, the value of tlag is about 0 h. The value tmax-tlag may be between about 2 and 3 hours. Further, the value tmax-tlag may be independent from the administered dosage of the glucocorticoid.

The delayed-release dosage form is advantageously administered together with or, e.g., not later than 3 h after a meal, e.g. during or upon 3 h after a meal.

Further, the present invention is described in more detail by the following figures and examples.

### FIGURE LEGENDS

FIG. 1 shows the pharmacokinetic profiles of Delayed-Release Prednisone and IR prednisone, Study NP01-013;

FIG. 2 shows pharmacokinetic profiles of Delayed-release Prednisone and IR prednisone, Study NP01-013, lag time corrected (tmax-tlag).

### EXAMPLES

Clinical Studies.

The clinical development program for the delayed-release Prednisone comprised 8 phase I studies and 2 phase III study:

Phase I Studies:
EMR 62215-001 and EMR 62215-002 were conducted to investigate the bioavailability and pharmacokinetic characteristics of experimental Delayed-Release Prednisone formulations with the aim to select a Delayed-Release Prednisone tablet with appropriate pharmacokinetic profile for evening administration.
EMR 62215-005 was conducted to compare the bioavailability and pharmacokinetic characteristics of Delayed-Release Prednisone (5 mg, administered in the evening) with immediate-release prednisone (5 mg, administered at 2 am).
NP01-006 evaluated the food effect.
NP01-008 evaluated the dose proportionality of 1 mg, 2 mg and 5 mg tablets.
NP01-009 and NP01-010 evaluated the bioavailability of batches with different in vitro lag times,
NP01-013 compared the bioavailability of Delayed-Release Prednisone (5 mg, administered at 10 pm after a light evening meal) and an IR prednisone formulation (5 mg, administered in the morning after breakfast).

Phase III Study:
NP01-007 (CAPRA-2) was a randomized, parallel-group, placebo-controlled, double-blind, phase 3 study in 350 adult RA subjects to evaluate if 12 weeks of treatment with 5 mg Delayed-Release Prednisone administered in the evening is superior to placebo.

Planned Study:
NP01-011 (CAPRA-3): Disease Progression Study:

Delayed-Release Prednisone is administered in the evening for 1, 2 or even 5 years. The daily prednisone dose could be between 1 to 10 mg/day, prefereably between 1 and 5 mg/day to investigate prevention from joint damage.

### Study Designs and Methodology

Pharmakokinetic Studies

NP01-006 (5 mg delayed-release prednisone; Food effect study): For prednisone no food effect has been reported in the literature. Prokein (1982) compared overnight fast vs. fed with 3 different diets and could not show any difference. He confirmed the findings from Tembo (1976) and Uribe (1976).

Surprisingly, in Study NP01-006 for Delayed-Release Prednisone a distinct effect of food on the oral bioavailability was shown.

In a study with 24 healthy subjects, oral absorption of prednisone from delayed-release prednisone was significantly affected by the intake of food. Under standard fasting conditions, both the maximum plasma concentration (Cmax) and the bioavailability of delayed-release prednisone were significantly lower than under fed conditions, shortly after intake of a high fat breakfast. The results are shown in Table 1. However, the amount of food and the timing of the meal relative to drug intake do not have an impact on the bioavailability of Delayed-Release Prednisone: both formulations where found to be bioequiValent when Delayed-Release Prednisone was taken 0.5 hour after a full meal or 2.5 hours after a light meal. Delayed-release prednisone thus should be taken not later than 3 h after a meal.

TABLE 1
Effect of Food on Delayed-Release Prednisone
Pharmacokinetics. Mean (SD)

| | | Delayed-Release | | Delayed-Release | |
|---|---|---|---|---|---|
| | | Prednisone | | Prednisone | |
| Prednisone | N | Fasted | | Fed | |
| Cₘₐₓ (ng/mL) | 24 | 6.6 | (3.7) | 19.1 | (3.2) |
| AUC₀₋ₗₐₛₜ (ng h/mL) | 24 | 34.2 | (21.9) | 100.8 | (18.7) |
| AUC_{0-∞} (ng h/mL) | 24 | 38.3 | (21.8) | 103.0 | (18.9) |
| t_{lag} (h) | 24 | 5.5 | (3.5-7.5) | 4.5 | (3.5-6.0) |
| tₘₐₓ (h) | 24 | 8.0 | (6.0-18.0) | 6.5 | (5.5-10.0) |
| t_{1/2} (h) | 24 | 2.6 | (1.1) | 2.5 | (0.5) |

tₘₐₓ and t_{lag} values are median (range)

NP01-013 compared the bioavailability of Delayed-Release Prednisone (5 mg, administered at 10 pm after a light evening meal) and Immediate Release prednisone (5 mg, administered in the morning after breakfast).

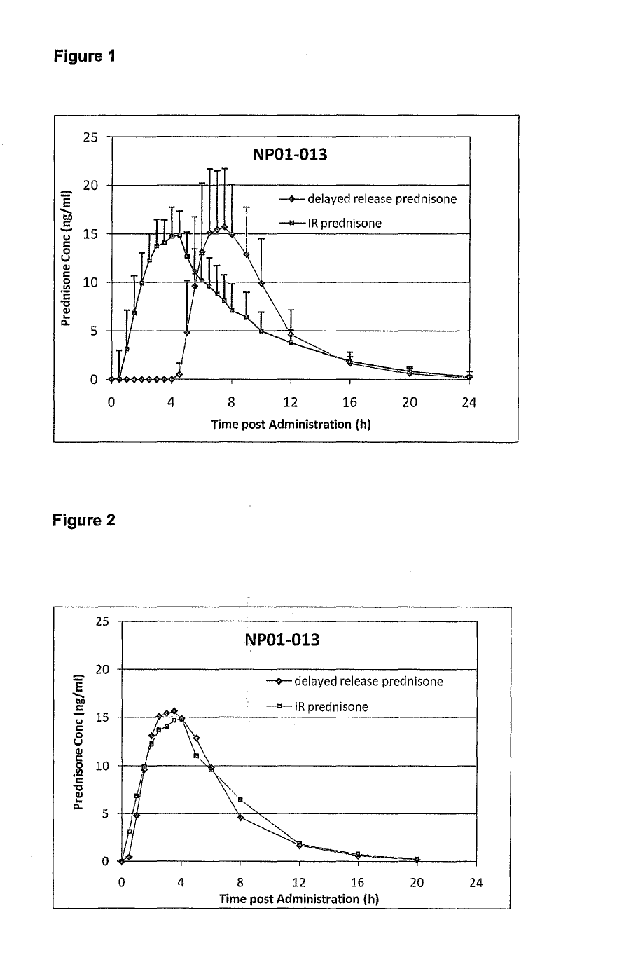

Surprisingly, the shape of the plasma-profile of both tablets, Delayed-Release Prednisone and Immediate release prednisone, were similar after the lag time has been achieved for the Delayed-Release Prednisone: Cmax, AUC and tmax-tlag were comparable. Tlag describes the lag time in vivo, Tmax describes the time until Cmax in reached. Surprisingly, tmax-tlag was for both Delayed-Release Prednisone and Immediate release prednisone about 2-4 hours. A further surprising finding was that the plasma profiles were identical under the concomitant administration of food. The results are shown in FIGS. 1 and 2.

NP01-007 (CAPRA-2)

Study Design:

CAPRA-2 was a randomized, multi-center, double-blind, parallel-group, placebo-controlled 13-week study conducted in 62 centers in 6 countries in Europe and North America. Patients with a history of rheumatoid arthritis (RA) who were on disease modifying anti-rheumatic drug (DMARD) treatment for RA for at least 6 months (with a stable dose for at least 6 weeks prior to the screening visit) and who had a duration of morning stiffness of at least 45 minutes were eligible for inclusion. Treatment with glucocorticoids other than the study medication was prohibited during the study. The study was composed of a single-blind 1-week screening phase and a 12-week double-blind treatment phase. During the screening phase all patients received placebo on top of their standard medication. No medication was withdrawn during this period, so patients remained treated at all times during the study. The screening phase included daily recording of duration of stiffness in diaries prior to Visit 1 (randomization visit) to calculate a robust baseline value. The double-blind treatment phase of the study started with Visit 1 (baseline; Week 0), when eligible patients were randomized to receive either Delayed-Release Prednisone (5 mg) or placebo at a fixed dose for 12 weeks. The double-blind treatment phase included 4 visits (Visit 1 to Visit 4; Weeks 0, 2, 6 and 12). The duration of the study for each patient was a maximum of 13 weeks (including the screening period). The primary analysis population was the safety population, which included all patients who were randomized and received at least 1 dose of study medication. Patients were analyzed according to the treatment which they actually received.

Objectives:

The objective was to evaluate if 12 weeks of treatment with 5 mg Delayed-Release Prednisone administered in the evening was superior to placebo in terms of the American College of Rheumatology (ACR) 20/50/70 response rate. Responders were defined as those whose improvement from baseline to endpoint (12 weeks) fulfilled all 3 of the following criteria:

(i) ≧20%, 50% or 70% reduction in the tender joint count (0-28).

(ii) ≧20%, 50% or 70% reduction in the swollen joint count (0-28).

(iii) ≧20%, 50% or 70% reduction in 3 of 5 of the following additional measures:
Patient assessment of pain (100 mm visual analogue scale [AS]).
Patient's global assessment of disease activity (VAS).
Physician's global assessment of disease activity (VAS).
Functional Disability Index of the Health Assessment Questionnaire (HAQ-DI).
C-reactive protein (CRP) or erythrocyte sedimentation rate (ESR) as acute-phase reactant. CRP was used. If the CRP result was not available then the ESR result was used to calculate the ACR20 responder status.

Another objective was to evaluate if 12 weeks of treatment with 5 mg Delayed-Release Prednisone administered in the evening was superior to placebo in terms of the relative reduction of morning stiffness.

Results: A total of 350 patients were enrolled and randomized to Delayed-Release Prednisone (231 patients) or placebo (119 patients). Overall, 323 patients completed the study (217 patients assigned to Delayed-Release Prednisone and 106 patients assigned to placebo) and 27 patients withdrew from the study (14 patients assigned to Delayed-Release Prednisone and 13 patients assigned to placebo). The most common primary reason for withdrawal was patient request (14 patients overall [4.0%]), followed by adverse event (AE; 7 patients overall [2.0%]).

The majority of patients were female (84.0%) and in the middle age group (>45 years to ≦65 years). Mean age was 57.2 years and mean duration of RA was 8.0 years. Treatment groups were comparable in terms of demographic and other baseline characteristics.

ACR 20 Responder Rate:

The proportion of responders increased with each visit in both treatment groups, but there were more responders in the Delayed-Release Prednisone group than in the placebo group at each visit (Table 2). At Visit 4 (Week 12), 47.2% of patients in the Delayed-Release Prednisone group were responders compared to 28.6% of patients in the placebo group (difference in proportions: 18.6% using worse case imputation). The odds ratio (2.31; confidence interval [Cl] 1.43, 3.75]) showed a significantly greater response rate for the Delayed-Release Prednisone group compared to the placebo group (p=0.0007).

TABLE 2
ACR20 Response by Treatment Group and Visit (Safety
Population, Worse Case)

Number (%) of patients

| | | Delayed-Release | |
|---|---|---|---|
| | ACR20 | Prednisone | Placebo |
| Visit | response | (N = 231) | (N = 119) |
| Visit 2 | Responders | 53 (22.9%) | 13 (10.9%) |
| | Non-responders | 170 (73.6%) | 101 (84.9%) |
| | Missing | 8 (3.5%) | 5 (4.2%) |
| Visit 3 | Responders | 94 (40.7%) | 29 (24.4%) |
| | Non-responders | 121 (52.4%) | 78 (65.5%) |
| | Missing | 16 (6.9%) | 12 (10.1%) |
| Visit 4 | Responders | 109 (47.2%) | 34 (28.6%) |
| | Non-responders | 115 (49.8%) | 82 (68.9%) |
| | Missing | 7 (3.0%) | 3 (2.5%) |

ACR 50 Responder Rate:

A summary of the ACR50 response is provided in Table 3. The proportion of responders according to the ACR50 criteria was greater in the Delayed-Release Prednisone group than in the placebo group at each visit. The highest response rate and the greatest difference between the treatment groups was seen at Visit 4, with a response rate of 22.5% in the Delayed-Release Prednisone group and 9.2% in the placebo group.

Whereas the response rate at baseline (Visit 2) was comparable in both treatment groups, there was a greater number of responders in the Delayed-Release Prednisone group than in the placebo group at Visit 3 and Visit 4, with a difference in proportions of 8.6% (p=0.0250) at Visit 3 and 13.5% (p=0.027) at Visit 4 for LOCF data.

TABLE 3
ACR50 Response (Safety Population)

Number (%) of patients

| | | Lodotra ™ | Placebo |
|---|---|---|---|
| Visit | ACR50 response | (N = 231) | (N = 119) |
| Visit 2 | Responders | 8 (3.5%) | 3 (2.5%) |
| | Non-responders | 216 (93.5%) | 111 (93.3%) |
| | Missing | 7 (3.0%) | 5 (4.2%) |
| Visit 3 | Responders | 33 (14.3%) | 7 (5.9%) |
| | Non-responders | 181 (78.4%) | 100 (84.0%) |
| | Missing | 17 (7.4%) | 12 (10.1%) |
| Visit 4 | Responders | 52 (22.5%) | 11 (9.2%) |
| | Non-responders | 171 (74.0%) | 105 (88.2%) |
| | Missing | 8 (3.5%) | 3 (2.5%) |

ACR 70 Responder Rate:

A summary of the ACR70 response is provided in Table 4. At Visit 2, there were only 0.9% responders according to ACR70 criteria in the Delayed-Release Prednisone group and 1.7% responders in the placebo group. Whereas the response rate remained nearly constant in the placebo group through Visit 4 (2.5%), the proportion of responders in the Delayed-Release Prednisone group increased continuously during the course of the study, reaching a response rate of 6.9% at Visit 4.

The difference in proportions between both treatment groups increased with each visit, reaching a 4.5% difference in favor of Delayed-Release Prednisone for LOCF data at Visit 4 (p=0.0955).

TABLE 4
ACR70 Response (Safety Population)

Number (%) of patients

| | ACR70 | Lodotra ™ | Placebo |
|---|---|---|---|
| Visit | response | (N = 231) | (N = 119) |
| Visit 2 | Responders | 2 (0.9%) | 2 (1.7%) |
| | Non-responders | 222 (96.1%) | 112 (94.1%) |
| | Missing | 7 (3.0%) | 5 (4.2%) |
| Visit 3 | Responders | 7 (3.0%) | 2 (1.7%) |
| | Non-responders | 210 (90.9%) | 105 (88.2%) |
| | Missing | 14 (6.1%) | 12 (10.1%) |
| Visit 4 | Responders | 16 (6.9%) | 3 (2.5%) |
| | Non-responders | 208 (90.0%) | 113 (95.0%) |
| | Missing | 7 (3.0%) | 3 (2.5%) |

Morning Stiffness:

The duration of morning stiffness at baseline was comparable in the Delayed-Release Prednisone group and the placebo group, with a total median duration of morning stiffness of 134.1 minutes (Table 5).

During the course of the study, the median duration of morning stiffness decreased in both treatment groups. However, the relative change in the median duration of morning stiffness was greater in the Delayed-Release Prednisone group than in the placebo group at each study visit; at the endpoint (Visit 4), the median duration of morning stiffness was 45.2 minutes in the Delayed-Release Prednisone group and 85.0 minutes in the placebo group, corresponding to a median relative change from baseline of −56.5% for the Delayed-Release Prednisone group and −33.3% for the placebo group.

At Visit 4, the Delayed-Release Prednisone group showed a significantly greater reduction in the duration of morning stiffness compared to the placebo group. The difference in the median relative change for LOCF data was −21.3 minutes (Cl 24.0, 17.4).

TABLE 5
Change in Duration of Morning Stiffness at Each Visit (Safety
Population, LOCF)

| | Duration of morning stiffness [min] | | | | | | |
|---|---|---|---|---|---|---|---|
| | Delayed-Release Prednisone | | | Placebo | | | |
| | (N = 231) | | | (N = 119) | | | Total |
| | | Absolute | Relative | | Absolute | Relative | (N = 350) |
| Visit | Value | change | change [%] | Value | change | change [%] | Value |
| Baseline | | | | | | | |
| (Visit 1) | | | | | | | |
| N | 231 | — | — | 119 | — | — | 350 |
| Mean | 152.7 | — | — | 155.4 | — | — | 153.6 |
| (SD) | (92.45) | | | (87.54) | | | (90.69) |
| Media | 128.6 | — | — | 138.6 | — | — | 134.1 |
| n | (0-473) | | | (40-395) | | | (0-473) |
| (range) | | | | | | | |
| Visit 2 | | | | | | | |
| N | 229 | 229 | 228 | 119 | 119 | 119 | — |
| Mean | 120.2 | −32.92 | −16.9 | 142.7 | −12.7 | −4.0 | — |
| (SD) | (97.19) | (74.27) | (95.67) | (93.49) | (64.38) | (48.85) | |
| Media | 90.0 | −28.6 | −22.2 | 126.4 | −10.0 | −8.1 | — |
| n | (0-456) | (−473-311) | (−100-1246) | (0-403) | (−261-175) | (−100-185) | |
| (range) | | | | | | | |
| Visit 3 | | | | | | | |
| N | 220 | 220 | 220 | 112 | 112 | 112 | — |
| Mean | 90.3 | −61.6 | −42.2 | 122.2 | −33.2 | −18.8 | — |
| (SD) | (94.70) | (81.15) | (47.72) | (97.24) | (78.26) | (53.40) | |
| Media | 60.0 | −53.6 | −49.8 | 103.2 | −24.3 | −24.2 | — |
| n | (0-416) | (−473-146) | (−100-180) | (0-380) | (−261-160) | (−100-162) | |
| (range) | | | | | | | |
| Visit 4 | | | | | | | |
| N | 216 | 216 | 216 | 107 | 107 | 107 | — |
| Mean | 86.0 | −65.2 | −43.9 | 114.1 | −39.5 | −20.6 | — |
| (SD) | (101.96) | (88.87) | (61.85) | (100.39) | (92.72) | (69.59) | |
| Media | 45.2 | −55.9 | −56.5 | 85.0 | −36.6 | −33.3 | — |
| n | (0-420) | (−473-154) | (−100-414) | (0-381) | (−335-274) | (−100-305) | |
| (range) | | | | | | | |

Safety Results:

Overall, 157 patients (44.9%) experienced at least 1 treatment-emergent adverse event (TEAE), with a slightly lower proportion in the Delayed-Release Prednisone group (42.9%) than in the placebo group (48.7%), as shown in Table 6. The most frequent TEAEs were related to worsening of RA such as arthralgia (13.7% of patients) and RA (7.4% of patients); these are consistent with the expected events for patients with RA.

TABLE 6
Summary of TEAEs Reported for ≧1% of all Patients by Preferred
Term (Safety Population)

| | Number (%) of patients | | |
|---|---|---|---|
| | Delayed- | | |
| | Release | | |
| | Prednisone | Placebo | Total |
| Preferred term | (N = 231) | (N = 119) | (N = 350) |
| Patients with any | 99 (42.9%) | 58 (48.7%) | 157 (44.9%) |
| TEAE | | | |
| Arthralgia | 24 (10.4%) | 24 (20.2%) | 48 (13.7%) |
| RA | 15 (6.5%) | 11 (9.2%) | 26 (7.4%) |
| Nasopharyngitis | 11 (4.8%) | 4 (3.4%) | 15 (4.3%) |
| Headache | 9 (3.9%) | 5 (4.2%) | 14 (4.0%) |
| Bronchitis | 3 (1.3%) | 5 (4.2%) | 8 (2.3%) |
| Hypertension | 5 (2.2%) | 1 (0.8%) | 6 (1.7%) |
| Rash | 4 (1.7%) | 1 (0.8%) | 5 (1.4%) |
| Diarrhea | 4 (1.7%) | 1 (0.8%) | 5 (1.4%) |
| Back pain | 3 (1.3%) | 1 (0.8%) | 5 (1.4%) |
| Hematuria | 1 (0.4%) | 3 (2.5%) | 5 (1.4%) |
| Vomiting | 3 (1.3%) | 1 (0.8%) | 5 (1.4%) |
| Odema peripheral | 2 (0.9%) | 2 (1.7%) | 5 (1.4%) |

Source: Section 14.3, 14.3.1.2.2 and Section 16, Listing 16.2.8.1.
N = total number of patients per treatment group and imputation scheme,
RA = rheumatoid arthritis,
TEAE = treatment-emergent adverse event.
A patient with multiple occurrences of a TEAE under 1 treatment was counted only once in the TEAE preferred term for that treatment.
TEAEs were coded using MedDRA version 11.0.

The incidence of treatment-related TEAEs was low (28 patients overall, 8.0%) and similar in both treatment groups; all treatment-related TEAEs occurred in <2% of patients in any treatment group. The most commonly reported treatment-related TEAE was headache (4 patients overall, 1.1%), followed by gastroesophageal reflux disease (0.6%), nausea (0.9%), peripheral edema (0.6%), hypercholesterolemia (0.6%) and insomnia (0.6%). Overall, 6 patients experienced TEAEs leading to withdrawal, 5 in the Delayed-Release Prednisone group and 1 in the placebo group. There were 3 patients (2 in the Delayed-Release Prednisone group and 1 in the placebo group) who withdrew due to headache; other TEAEs leading to withdrawal were experienced by no more than 1 patient for any TEAE. In addition, there were 9 patients in the Delayed-Release Prednisone group who experienced 14 other significant TEAEs (TEAEs that were not listed as known side effects of Delayed-Release Prednisone/prednisone but were considered to be treatment-related in this study) that were all mild (8 TEAEs) or moderate (10 TEAEs) in severity. None of the other significant TEAEs was serious.

Serious adverse events (SAEs) during the double-blind treatment phase were reported for 1 patient treated with Delayed-Release Prednisone and 2 patients treated with placebo, All SAEs were considered to be not related to study treatment. No deaths occurred during this study.

The majority of TEAEs were mild in severity. Severe TEAEs were reported for 3 patients (1.3%) in the Delayed-Release Prednisone group and 5 patients (4.2%) in the placebo group. There were no patients with severe SAEs and only 1 patient (0.8%) with a severe TEAE leading to withdrawal in the placebo group.

There were no safety concerns in terms of laboratory safety, physical examination and vital signs.

Further prevention of structural damages on the joints may be more pronounced with the treatment of the Delayed Release Prednisone because of the surprising effects on the bone turnover.

Osteocalcin

Osteoporosis is a known side effect of glucocorticoids, therefore in CAPRA-2, the concentration of the bone formation marker osteocalcin was determined at screening and Visit 4. Results are summarized in Table 7. The mean osteocalcin concentration was similar for the Delayed-Release Prednisone group and the placebo group at screening (total mean concentration 6.76 ng/mL [SD 3.171 ng/mL]). Surprisingly, there was no clinically notable change in mean osteocalcin concentration between screening and Visit 4 for the Delayed-Release Prednisone group while under placebo osteocalcin increased.

TABLE 7
Summary of Osteocalcin Concentration (Safety Population)

| | | Osteocalcin [ng/mL] | | |
|---|---|---|---|---|
| | | Delayed- | | |
| | | Release | | |
| | | Prednisone | Placebo | Total |
| Visit | Statistic | (N = 231) | (N = 119) | (N = 350) |
| Screening | N | 224 | 116 | 340 |
| (Visit 0) | Mean (SD) | 6.78 (3.251) | 6.70 (3.023) | 6.76 (3.171) |
| | Median | 6.20 | 6.35 | 6.30 |
| | (range) | (0.3-23.3) | (0.3-23.1) | (0.3-23.3) |
| Visit 4 | N | 218 | 110 | 328 |
| | Mean (SD) | 6.61 (3.184) | 7.95 (4.027) | 7.06 (3.540) |
| | Median | 6.10 | 7.70 | 6.40 |
| | (range) | (0.3, 21.8) | (0.3, 25.1) | (0.3, 25.1) |

Urine CTX I

Urine CTX I is a marker for bone degradation. As bone degradation and inare considered to be tightly related in inflammatory diseases such as RA, urine CTX I concentrations were evaluated at screening and Visit 4.

A summary of urine CTX I (observed case) is provided in Table 8. The mean urine CTX I concentration at screening was comparable in the Lodotra group and the placebo group, with a total mean urine CTX I concentration of 195.9 μg/mmol Cr. At Visit 4, the mean urine CTX I concentration had increased compared to screening in both treatment groups, with a surprisingly smaller mean absolute increase in the Lodotra group (76.4 μg/mmol Cr) than in the placebo group (144.5 μg/mmol Cr).

TABLE 8
Urine CTX I (Safety Population)

| | | Urine CTX I [μg/mmol Cr)] | | | | |
|---|---|---|---|---|---|---|
| | | Lodotra | | Placebo | | |
| | | (N = 231) | | (N = 119) | | Total |
| | | | Absolute | | Absolute | (N = 350) |
| Visit | Statistic | Value | change | Value | change | Value |
| Screening | n | 177 | — | 90 | — | 267 |
| (Visit 0) | Mean | 183.7 | — | 220.1 | — | 195.9 |
| | (SD) | (221.25) | | (262.74) | | (236.19) |
| | Median | 112.0 | — | 106.0 | — | 108.0 |
| | (range) | (11-1759) | | (11-1260) | | (11-1759) |
| Visit 4 | n | 173 | 142 | 98 | 79 | — |
| | Mean | 263.5 | 76.4 | 346.8 | 144.5 | — |
| | (SD) | (371.87) | (455.02) | (634.06) | (709.95) | |
| | Median | 145.0 | 17.0 | 168.0 | 46.0 | — |
| | (range) | (9, 2818) | (−1361, | (16, 4578) | (−1165, | |
| | | | 2757) | | 4018) | |

High levels of urinary CTX-1 predict an increased risk of radiologic progression in patients with RA, especially those without radiologic joint damage present (Garnero 2002).

Conclusion:

Surprisingly, with Delayed-Release Prednisone administered at about 22:00 bone turnover and bone degradation are reduced compared to Placebo while for prednisone administered in the morning a negative effect on osteoporosis and bone metabolism is described in the literature.

Maximum plasma levels of prednisone in the early morning hours (starting at 02:00) are obtained by administration of Prednisone delayed-release at about 22:00 which is an acceptable time for the patient.

Prednisone delayed-release tablets can be used in patients with severe, moderate or mild rheumatoid arthritis.

Prednisone Delayed-Release can be used in patients with short, mid-term or long-lasting disease duration.

Prednisone Delayed-Release can be used in patients with rheumatoid arthritis to reduce signs and symptoms.

Prednisone Delayed-Release can be used in patients with minor or incomplete clinical response to other medicaments.

Prednisone Delayed-Release can be used in patients to achieve remission.

Prednisone Delayed-Release can be used in patients before, at an early or advanced stage of damage of the joints.

Prednisone delayed-release tablets can be used in patients pre-treated with corticosteroids, in those who are refractory to treatment or in corticoid naïve patients.

Prednisone delayed-release tablets can be used as monotherapy or more likely in combination with DMARDs, NSAIDs, TNF α Inhibitors and/or analgetics.

Prednisone delayed-release tablets can be used for short, mid or long-term treatment.

### LITERATURE REFERENCES

ACR (American College of Rheumatology) Subcommittee on Rheumatoid

Arthritis Guidelines. Guidelines for the management of rheumatoid arthritis. Arthritis Rheum 2002; 46:328-46.

Ahlmén M, Nordenskiöld U, Archenholtz B, Thyberg I, Rönnqvist R, Lindén L, et al. Rheumatology outcomes: the patient's perspective. A multicentre focus group interview study of Swedish rheumatoid arthritis patients. Rheumatology 2005; 44:105-10.

Arnett F C, Edworthy S M, Bloch D A, McShane D J, Fries J F, Cooper N S, et al. The American Rheumatism Association 1987 revised criteria for the classification of rheumatoid arthritis. Arthritis Rheum 1988; 31:315-24.
Arvidson N G, Gudbjörnsson B, Elfman L, Ryden A C, Tötterman T H, Hällgren R. Circadian rhythm of serum interleukin-6 in rheumatoid arthritis. Ann Rheum Dis 1994; 53:521-4.
Arvidson N G, Gudbjörnsson B, Larsson A, Hällgren R. The timing of glucocorticoid administration in rheumatoid arthritis. Ann Rheum Dis 1997; 56:27-31.

Bijlsma J W J, Boers M, Saag K G, Furst D E. Glucocorticoids in the treatment of early and late R A. Ann Rheum Dis 2003; 62:1033-7.
Bijlsma J W J, Saag, K G, Buttgereit F, da Silva J A P. Developments in glucocorticoid therapy. Rheum Dis Clin N Am 2005; 31:1-17.
Boers M. Glucocorticoids in rheumatoid arthritis: a senescent research agenda on the brink of rejuvenation? Best Practice Research Clin Rheumatol 2004; 18 (1):21-9.
Buttgereit F, Burmester G-R, Lipworth B J. Optimised glucocorticoid therapy: the sharpening of an old spear. Lancet 2005; 365:801-3.
Buttgereit F, Saag K G, Cutolo M, da Silva J A P, Bijlsma J W J. The molecular basis for the effectiveness, toxicity, and resistance to glucocorticoids: focus on the treatment of rheumatoid arthritis. Scand J Rheumatol 2005; 34:14-21.
Buttgereit F, Straub R H, Wehling M, Burmester G R. Glucocorticoids in the treatment of rheumatic diseases: an update on the mechanisms of action. Arthritis Rheum 2004; 50:3408-17.
Buttgereit F, Doering G, Schaeffler A, Witte S, Sierakowski S, Gromnica-Ihle E, Jeka S, Krueger K, Szechinski J, Alten R: Efficacy of modified-release versus standard prednisone to reduce duration of morning stiffness of the joints in rheumatoid arthritis (CAPRA-1): a double-blind, randomized controlled trial; Lancet 2008; 371: 205-14.
Capell H A, Madhok R, Hunter J A, Porter D, Morrison E, Larkin J, et al. Lack of radiological and clinical benefit over two years of low dose prednisolone for rheumatoid arthritis: results of a randomised controlled trial. Ann Rheum Dis 2004; 63:797-803.
Carr A, Hewlett S, Hughes R, Mitchell H, Ryan S, Carr M, et al. Rheumatology outcomes: the patient's perspective. J Rheumatol 2003; 30:880-3.
Conn D L. Resolved: Low-dose prednisone is indicated as a standard treatment in patients with rheumatoid arthritis. Arthritis Rheum 2001; 45:462-7.
Crofford L J, Kalogeras K T, Mastorakos G, Magiakou M A, Wells J, Kanik K S, et al. Circadian relationships between interleukin (IL)-6 and hypothalamicpituitary-adrenal axis hormones: failure of IL-6 to cause sustained hypercortisolism in patients with early untreated rheumatoid arthritis. J Clin Endocrinol Metab 1997; 82:1279-83.
Cutolo M, Maestroni G J M, Otsa K, Aakre O, Villaggio B, Capellino S, et al. Circadian melatonin and cortisol levels in rheumatoid arthritis patients in winter time: a north and south Europe comparison. Ann Rheum Dis 2005; 64:212-6.
Cutolo M, Masi A T. Circadian rhythms and arthritis. Rheum Dis Clin N Am 2005; 31:115-29.
Cutolo M, Seriolo B, Craviotto C, Pizzorni C, Sulli A. Circadian rhythms in RA. Ann Rheum Dis 2003; 62:593-6.
Da Silva J A P, Jacobs J W G, Kirwan J R, Boers M, Saag K G, Ines L B S, et al. Safety of low dose glucocorticoid treatment in rheumatoid arthritis: published evidence and prospective trial data. Ann Rheumatol Dis 2006; 65:285-93. Garnero PLandewe R, Boers M, Verhoeven A, van der Linden S, Christgau S, van der Heijde D, Boonen D, Geusens P. Association of Baseline Levels of Markers of Bone and Cartilage Degradation With Long-Term Progression of Joint Damage in Patients With Early Rheumatoid Arthritis; Arthritis Rheumatism 2002, 46, 11, pp 2847
Gudbjörnsson B, Skogseid B, Öberg K, Wide L, Hällgren R. Intact adrenocorticotropic hormone secretion but impaired cortisol response in patients with active rheumatoid arthritis. Effect of glucocorticoids. J Rheumatol 1996; 23:596-602.
Guidance for Industry “Clinical Development Programs for Drugs, Devices, and Biological Products for the Treatment of Rheumatoid Arthritis (RA); FDA, February 1999.
Heshmati H M, Riggs B L, Burritt M F, McAlister C A, Wollan P C, Khosla S. Effects of the circadian variation in serum cortisol on markers of bone turnover and calcium homeostasis in normal postmenopausal women. J Clin Endocrinol Metab 1998; 83:751-6.
Hewlett S, Carr M, Ryan S, Kirwan J, Richards P, Carr A, et al. Outcomes generated by patients with rheumatoid arthritis: How important are they? Musculoskeletal Care 2005; 3:131-42.
Hickling P, Jacoby R K, Kirwan J R. Joint destruction after glucocorticoids are withdrawn in early rheumatoid arthritis. Arthritis and Rheumatism Council Low Dose Glucocorticoid Study Group. Br J Rheumatol 1998; 37:930-6.
Hudson M, Baron M. Morning stiffness is a better predictor of function in early inflammatory arthritis than are swollen and tender joints. Arthritis Rheum 2005; 52 Suppl 9: abstract 1036.
Jacobs J W G, van Everdingen A A, Verstappen S M M, Bijlsma J W J. Followup radiographic data on patients with rheumatoid arthritis who participated in a two-year trial of prednisone therapy or placebo. Arthritis Rheum 2006; 54:1422-8.
Karatay S. et al, The timing of low dose glucocorticoid therapy in the treatment of rheumatoid arthritis, The Pain Clinic, 2002, 13, 4, 305-312
Kirwan J R. The effect of glucocorticoids on joint destruction in rheumatoid arthritis. The Arthritis and Rheumatism Council Low-Dose Glucocorticoid Study Group. N Engl J Med 1995; 333:142-6.
Kirwan J R, Boers M, Shea B. Glucocorticoids strongly suppress joint damage in rheumatoid arthritis: A meta-analysis of 1,414 patients in 15 trials. Arthritis Rheum 2005; 52 Suppl 9: abstract 891.
LeLoet X et al, Clinical practice decision tree for the coice of the first disease modifying antirheumatic drug for very early rheumatoid arthritis: a 2004 proposal of the French Society of Rheumatology, Annals of Rheum Dis 2006; 65:45-50
Mastorakos G, Ilias I. Relationship between interleukin-6 (IL-6) and hypothalamic-pituitary-adrenal axis hormones in rheumatoid arthritis. Z Rheumatol 2000; 59(Suppl 2):75-9.
Petrovsky N, McNair P, Harrison L C. Diurnal rhythms of pro-inflammatory cytokines: regulation by plasma cortisol and therapeutic implications. Cytokine 1998; 10:307-12.
Pincus T, Sokka T, Kautiainen H. Patients seen for standard rheumatoid arthritis care have significantly better articular, radiographic, laboratory, and functional status in 2000 than in 1985. Arthritis Rheum 2005; 52:1009-19.
Prokein R. (Doctoral Thesis), E. Merck Darmstadt, Germany, 1982
Saag K G, Criswell L A, Sems K M, Nettleman M D, Kolluri S. Low-dose corticosteroids in rheumatoid arthritis—A meta-analysis of their moderate-term effectiveness. Arthritis Rheum 1996; 39:1818-25.
Saag K G, Koehnke R, Caldwell J R, Brasington R, Burmeister L F, Zimmerman B, et al. Low dose long-term corticoid therapy in rheumatoid arthritis: an analysis of serious adverse events. Am J Med 1994; 96:115-23.
Stucki G, Cieza A. The international classification of functioning, disability and health (ICF) core sets for rheumatoid arthritis: a way to specify functioning. Ann Rheum Dis 2004; 63(Suppl 2):40-5.
Svensson B, Boonen A, Albertsson K, van der Heijde D, Keller C, Hafstrom J. Low-dose prednisolone in addition to the initial disease-modifying antirheumatic drug in patients with early active rheumatoid arthritis reduces joint destruction and increases the remission rate: a two-year randomized trial. Arthritis Rheum 2005; 52:3360-70.
Tembo A V et al., J Clin Pharmacol 16, 1976: 620
Uribe M et al., Gastroenterology 71, 1976: 362
van Everdingen A A, Jacobs J W G, Siewertsz van Reesema D R, Bijlsma J W J. Low-dose prednisone therapy for patients with early active rheumatoid arthritis: clinical efficacy, disease-modifying properties, and side effects. A randomized, double-blind, placebo-controlled clinical trial. Ann Intern Med 2002; 136:1-12.
van Staa T P, Leufkens H G M, Abenhaim L, Zhang B, Cooper C. Oral corticosteroids and fracture risk: relationship to daily and cumulative doses. Rheumatology 2000; 39:1383-9.
Wassenberg S, Rau R, Steinfeld P, Zeidler H. Very low-dose prednisolone in early rheumatoid arthritis retards radiographic progression over two years. Arthritis Rheum 2005; 52:3371-80.

1-47. (canceled)

48. A method of administering prednisone to a patient having rheumatoid arthritis with a pain level of >50-70 mm on a 100 mm self reported visual analogue scale for pain comprising
administering an oral dosage form comprising an initial dosage of 1, 2, 5, or 10 mg of prednisone, to the patient once daily at or before bedtime, wherein the oral dosage form is administered with food,
determining the patient's clinical response to said administration, and
if said patient has a satisfactory clinical response, the method further comprises administering a maintenance dosage that is less than the initial dosage until a dosage of prednisone which will maintain a satisfactory clinical response is reached, or
if said patient does not have a satisfactory clinical response, the method further comprises administering an increased dosage that is more than the initial dosage until a satisfactory clinical response is determined,
wherein release of the prednisone from the oral dosage form is delayed for a time period (tlag) of 2-10 hours after administration of the oral dosage form.

49. The method of claim 48, wherein the patient also is being administered a disease modifying anti-rheumatic drug.

50. The method of claim 48, wherein the dosage form comprises 10 mg prednisone.

51. The method of claim 48, wherein the dosage form comprises 5 mg prednisone.

52. The method of claim 48, wherein the dosage form comprises 2 mg prednisone.

53. The method of claim 48, wherein the dosage form comprises 1 mg prednisone.

54. The method of claim 48, wherein the delayed-release dosage form has a tlag of from about 2 hours to about 6 hours after administration.

55. The method of claim 48, wherein the delayed-release dosage form has a tlag of from about 3 hours to about 5 hours after administration.

56. The method of claim 48, wherein the food is a high-fat meal.
